(19) United States
(12) Patent Application Publication
(10) Pub. No.: US 2015/0094675 A1
(43) Pub. Date: Apr. 2, 2015

(54) Stoma Stopper Securement Dressing

(71) Applicant: TIDI Securement Products, LLC, Neenah, WI (US)

(72) Inventors: Kurt Kyvik, Ocala, FL (US); Scott Ryan, Ocala, FL (US); Arthur Parkhurst, Ocala, FL (US)

(21) Appl. No.: 14/043,093

(22) Filed: Oct. 1, 2013

(51) Int. Cl. A61F 5/44; A61F 5/449; A61F 5/443

(52) U.S. Cl. CPC A61F 5/4407; A61F 5/443; A61F 5/449
USPC 604/337

(57) ABSTRACT

A stoma stopper securement dressing having a base member formed of a thin, flexible, skin-conforming sheet material having an adhesive coating on its underside for securing the base member to a skin surface, an opening in the base member to allow for passage of a stopper shaft therethrough, and a thin, flexible, releasable strap member attached to the base member, one end of the strap member being fixed to the based member and the other end of the strap member being a free end, such that the strap member can be opened to expose the opening for insertion or removal of a stoma stopper and can be closed to retain the stoma stopper therein.

### BACKGROUND OF THE INVENTION

This invention relates generally to the field of devices, dressings, bandages or the like applied to the human body to cover or seal a wound, injury or other opening in the skin surface, and more particularly to such devices that are adhesively affixed to the skin surface and utilized to secure a stopper, plug or similar blockage member inserted into a stoma or other bodily opening, and even more particularly to such devices that releasably retain the stopper within the stoma, such that the device may be opened to allow for removal and reinsertion of the stopper while the device remains affixed to the skin surface.

For certain medical conditions it is necessary to create passages or tunnels through the skin surface and into the underlying tissues, the passages leading to internal organs or cavities, such that liquids, medicines, gases, etc. may be delivered internally or released through catheters, cannulas or the like. The opening at the skin surface for such a passage is referred to as a stoma. For example, for individuals that cannot properly evacuate their bowels, a procedure known as antegrade colonic enema (ACE) has been developed. In an ACE procedure a passage is formed through the abdominal skin surface and tissue into the colon, such that a catheter may be inserted through the stoma and into the bowel to deliver a washout fluid to flush the bowels when needed. Other medical procedures that involve the formation of stomas include for example Mitronaff, caecostomy and Monti procedures.

It is often necessary for the stoma to be removably sealed, closed or covered when the catheter or other delivery member is not positioned within the passage, and stoppers, plugs or similar blocking members are known for this purpose, the stoppers being inserted a short distance into the passageway through the stoma. In a basic form, the stopper comprises an elongated shaft joined to a head of significantly greater diameter than the shaft. The shaft of the stopper is inserted through the stoma and the head rests on the skin surface, the diameter of the head also being significantly greater than the diameter of the stoma so as to remain on the skin surface to limit the insertion depth of the stopper and provide a member easily gripped for removal. Insertion of the stopper serves to preclude leakage from the passageway, reduce contamination or infection, and/or to maintain the stoma and passageway in an open status by preventing undesired tissue healing.

Examples of various sealing devices and systems are shown in U.S. Pat. No. 3,565,073 to Giesy, U.S. Pat. No. 4,121,589 to McDonnell, U.S. Pat. No. 4,261,340 to Baumel et al., U.S. Pat. No. 4,344,433 to Smith, U.S. Pat. No. 4,863,438 to Gauderer et al., U.S. Pat. No. 4,941,869 to D'Amico, U.S. Pat. No. 4,981,465 to Ballan et al., U.S. Pat. No. 5,045,052 to Sans, U.S. Pat. No. 5,125,916 to Panebianco et al., U.S. Pat. No. 6,485,476 to von Dyck et al., U.S. Pat. No. 6,668,831 to Hegwood, U.S. Pat. No. 6,723,079 to Cline, and U.S. Pat. No. 7,678,082 to Oishi et al. Many of these devices involve relatively rigid caps, large plugs or complicated locking structures that occupy a relatively large footprint and protrude an undesirable distance from the skin surface, such that the devices are not completely hidden by clothing or are difficult or cumbersome to open and close.

A simple method for securing the stopper within a stoma is to place an adhesive dressing or bandage over the stopper head. While this presents the desirable low profile, problems such as skin irritation or damage will occur due to the repeated removal and replacement of the adhesive dressing, as it is not uncommon in many cases for the stopper to be removed and replaced several times per day.

It is an object of this invention to provide an stoma stopper securement dressing in the form of a low profile, flexible, skin-conforming, bandage-like stoma covering that may be adhered to the skin and left in place during repeated removals and insertions of the stopper in order to minimize skin irritation or damage, the dressing having an open status allowing access to the stopper and a closed status wherein the stopper is maintained in position within the stoma. It is a further object to provide such a dressing that is flexible and conforming to the shape of the skin surface surrounding the stoma, the overall height of the dressing being of minimal dimension.

### SUMMARY OF THE INVENTION

The invention is in general a stoma stopper securement dressing that remains adhered to the skin during repeated insertions and removals of the stopper, the dressing having a low profile and formed as a flexible, skin-conforming structure. The dressing comprises a thin base member formed of a flexible, skin-conformable sheet material and having adhesive undercoating, and the term “dressing” is used herein to particularly distinguish the invention from a stoma cover composed of a relatively rigid, fixed-shape body, such as a plastic cap. An opening is generally centered in the base member of the dressing to allow passage therethrough of the shaft of a stopper, the opening being of smaller diameter than the diameter of the head of the stopper to be utilized with the dressing. A strap member formed of a flexible, conformable sheet material has a fixed end attached to the upper side of the base member, the strap member extending across and over the opening. The free end of the strap member is releasably connected to the base member by a fastener member or system. The fastener system preferably comprises a hook-and-loop type fastener, such that the hook elements of the fastener are mounted onto the upper surface of the base member and the loop members are mounted or formed integrally on the underside of the strap member free end, although it is understood that a releasable adhesive that allows for multiple detachments and reattachments would also be suitable.

With this structure, the stoma stopper securement dressing is adhered to the skin surface with the base member opening positioned on the stoma. With the strap member released and pulled away from the base member, the shaft of the stopper may be inserted through the opening and into the stoma. The head of the stopper, preferably configured as a thin disk, now sits on the upper surface of the base member. The strap member is then drawn back across the stopper head and refastened to the base member, thereby precluding movement of the stopper. To remove the stopper, the free end of the strap is disconnected from the base member and pulled away from the base member to expose the stopper head for removal. In this manner, repeated insertions and withdrawals of the stopper may be accomplished without requiring removal and replacement of the securement dressing itself.

Alternatively described, the invention is a stoma stopper securement dressing, alone or in combination with a stoma stopper, comprising a base member composed of a thin, flexible, skin-conformable sheet material, said base member having an upper surface and an underside, said underside being provided with an adhesive coating, said base member further comprising a centralized opening; a strap member having a fixed end affixed to said base member and a free end, said strap member composed of a thin, flexible, conformable sheet material, said free end of said strap member being releasably attached to said base member, said strap member having a closed status when said free end is attached to said base member and an open status when said free end of said strap member is detached from said base member, said strap member extending across said opening in said base member when in said closed status.

### BRIEF DESCRIPTION OF THE DRAWINGS

FIG. 1 is a perspective view of an embodiment of the securement dressing and a representative stopper, shown with the strap member in the open status.

FIG. 2 is a top view of the securement dressing of FIG. 1, shown in the closed status with the stopper head retained under the strap member.

FIG. 3 is a cross-sectional view of the securement dressing of FIG. 1, shown in the closed status as adhered to the skin surface, the stopper shaft extending through the stoma and passage.

FIG. 4 is a perspective view of an alternative embodiment of the securement dressing and a representative stoma stopper, shown in the open status with the stopper inserted into the stoma.

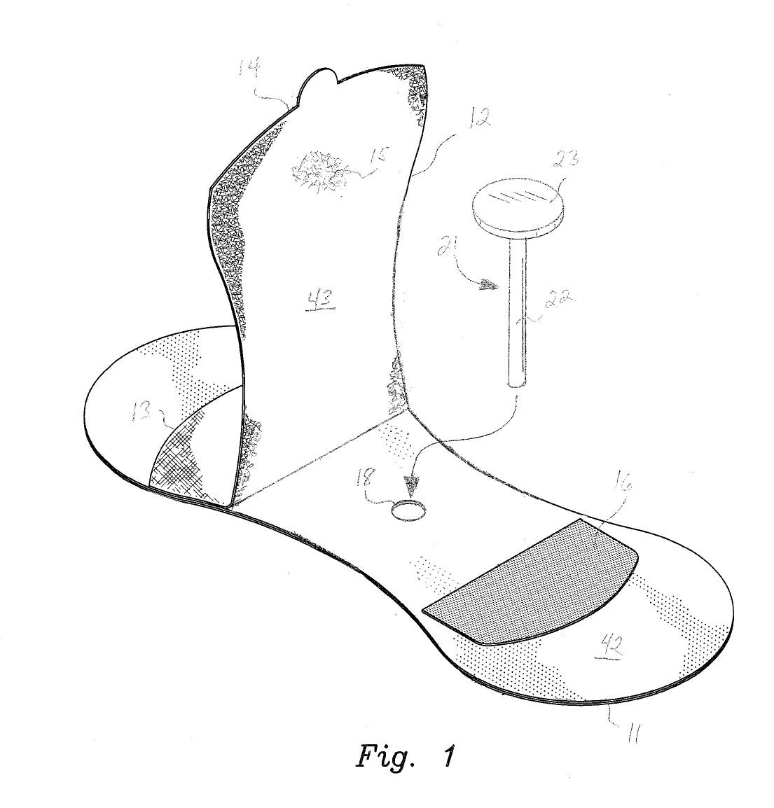

### DETAILED DESCRIPTION OF THE INVENTION

With reference to the drawings, the invention will now be described in detail. The invention is in general a stoma stopper securement dressing that remains adhered to the skin surface 31 during repeated insertions and removals of the stopper 21, the dressing having a low profile and formed as a flexible, conforming structure. The term “dressing” as used herein shall be taken to refer to a thin, bandage-like, stoma covering having a low profile and formed of a flexible, skin-conforming material or materials, as opposed to and distinct from stoma covering devices formed of a semi-rigid polymer material possessing three-dimensional structural stability, such as cap-type stoma covers utilizing interlocking or mechanical releasable closure mechanisms.

In circumstances where an internal passage 33 is formed through tissue 32 and an opening or stoma 34 is formed at the skin surface 31, it is often required to utilize a stopper, plug or similar instrument to block the stoma 34 when the passage 33 is not being used, as shown in FIG. 3. A typical stopper 21 comprises an elongated shaft 22, the length and dimensions of which may vary, adjoined to a head 23. The head 23 is significantly larger in width or diameter than the width or diameter of the shaft 22 in order to define an insertion stop when the shaft 22 is inserted through the stoma 34. The head 23 also presents a structure that may be gripped by the user in order to withdraw the stopper 21 from the stoma 34. Preferably, the head 23 is a thin, disk-shaped member so as to present a low profile.

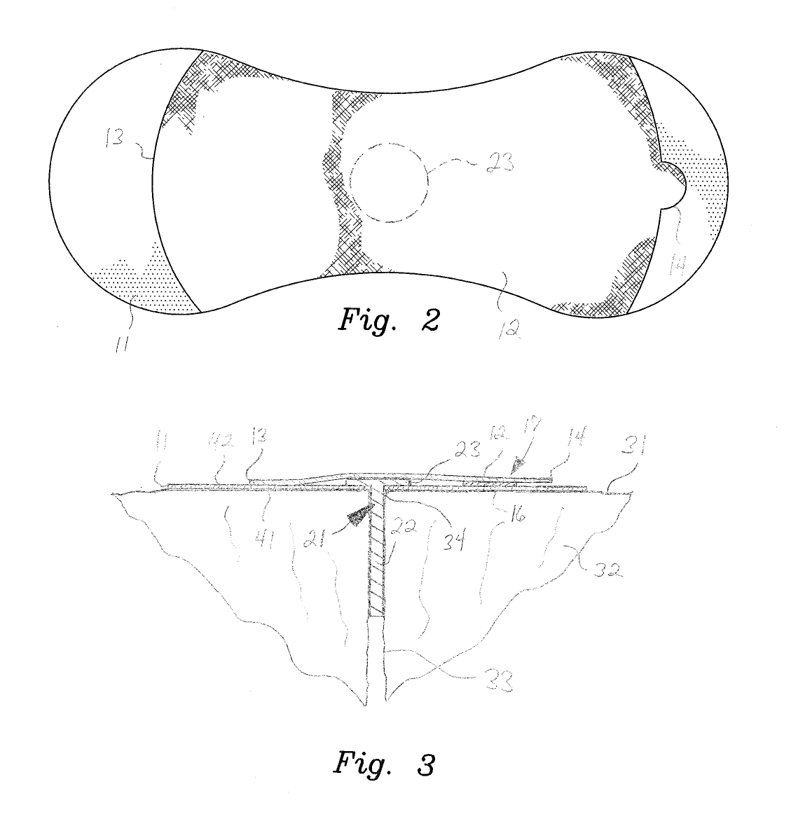

As shown in FIGS. 2 and 3, the securement dressing comprises a thin base member 11 formed of a flexible, skin-conformable sheet material and having an adhesive undercoating on its underside 41, the adhesive being one suitable for long-term compatibility with the skin surface 31. Material of the type found in typical adhesive bandages, such as for example a polymer or fabric sheet material, is a suitable material of construction for the base member 11, as are the typical adhesives utilized in bandages. An opening 18, preferably circular, is generally centered in the base member 11 to allow passage therethrough of the shaft 22 of the stopper 21, the opening 11 also preferably being of smaller diameter or width than the diameter or width of the head 23 of the stopper 21. With this sizing the head 23 will be prevented from passing through the opening 18 in the base member 11 such that a portion of the base member upper surface 42 remains disposed between the stopper head 23 and the skin surface 31.

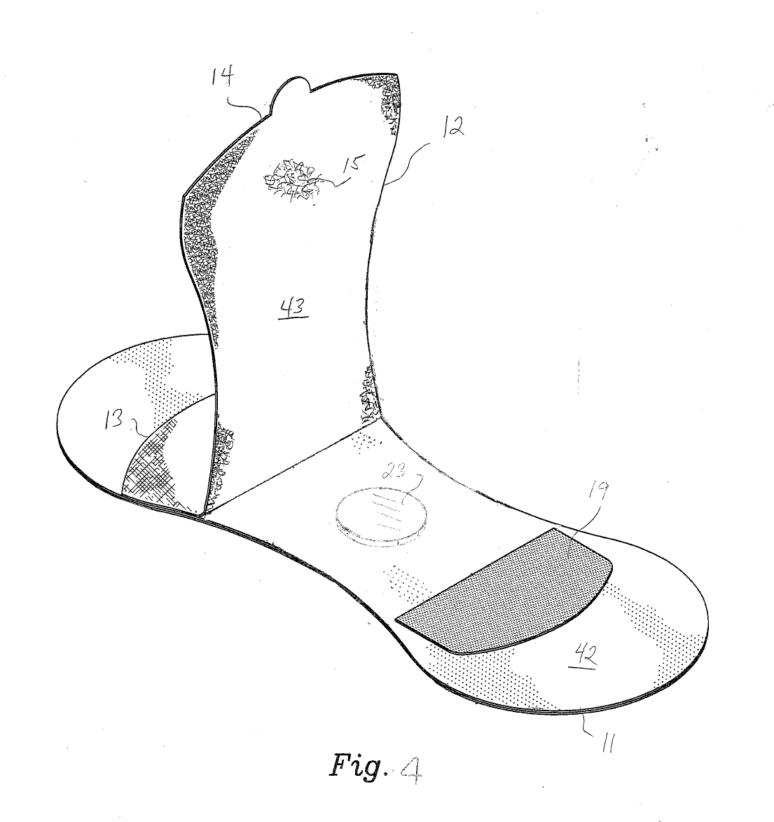

A thin, flexible strap member 12 formed of a flexible, skin-conformable sheet material, such as a polymer or fabric, has a fixed end 13 attached to the upper surface 42 of the base member 11 in suitable manner, such as with an adhesive, stitching, bonding, etc. The strap member 12 is a substantially planar member in its relaxed, non-tensioned state. The strap member 12 is sized and oriented so as to extend across and over the opening 18, the width of the strap member 12 being preferably wider than the stopper head 23. The free end 14 of the strap member 12 is releasably connected to the base member 11 by a fastener member or system 17. The fastener system 17 preferably comprises a hook-and-loop type fastener, such that the hook elements 16 of the fastener 17 are mounted onto the upper surface 42 of the base member 11 and the loop members 15 are mounted or formed integrally on the underside 43 of the strap member free end 14. The term integrally formed is meant to include herein choosing a material of construction for the strap member 12 that inherently serves as the loop elements 15 of the hook-and-loop fastener 17 when brought into contact with the hook elements 16. Alternatively the free end 14 of the strap member 12 may be releasably joined to the base member upper surface 42 by a suitable pressure sensitive adhesive 19 that allows for multiple detachments and reattachments, as shown in FIG. 4.

With this structure, the stoma stopper securement dressing may be adhered to the skin surface 31 with the base member opening 18 positioned surrounding the stoma 34, as shown in FIG. 3. In an open status with the strap member free end 14 released and pulled away from the base member 11, the shaft 22 of the stopper 21 may be inserted through the opening 18 and through the stoma 34 into the passage 33. The head 23 of the stopper 21, preferably configured as a thin disk, now sits on the upper surface 42 of the base member 11. The strap member 12 is then positioned back across the stopper head 23 and refastened to the base member 11 in its closed status, thereby precluding movement of the stopper 21. Because the strap member 12 and base member 11 are relatively thin, and further because the stopper head 23 is preferably relatively thin, a low profile is presented that does not excessively protrude or extend from the skin surface 31. To remove the stopper 21, the free end 14 of the strap 12 is disconnected from the base member 11 and pulled away from the base member 11 into the open status to expose the stopper head 23 for easy removal. In this manner, repeated insertions and withdrawals of the stopper 21 may be accomplished without requiring removal and replacement of the securement dressing itself, thereby minimizing irritation and damage to the underlying skin surface 31.

It is understood that substitutions and equivalents for certain elements set forth above may be obvious to those of skill in the art, and therefore the true scope and definition of the invention is to be as set forth in the following claims.

We claim:

1. A stoma stopper securement dressing comprising:
a base member composed of a thin, flexible, skin-conformable sheet material, said base member having an upper surface and an underside, said underside being provided with an adhesive coating, said base member further comprising a centralized opening;
a strap member having a fixed end affixed to said base member and a free end, said strap member composed of a thin, flexible, conformable sheet material, said free end of said strap member being releasably attached to said base member, said strap member having a closed status when said free end is attached to said base member and an open status when said free end of said strap member is detached from said base member, said strap member extending across said opening in said base member when in said closed status.

2. The dressing of claim 1, wherein said strap member fixed end is affixed to said upper surface of said base member.

3. The dressing of claim 1, wherein said free end of said strap member is releasably attached to said upper surface of said base member by a hook-and-loop fastener.

4. The dressing of claim 3, wherein said hook-and-loop fastener comprises hook elements mounted on said upper surface of said base member and wherein said strap member sheet material comprises loop elements.

5. The dressing of claim 1, wherein said strap member is a substantially planar member when non-tensioned.

6. A stoma stopper securement dressing in combination with a stoma stopper comprising:
a stoma stopper comprising an elongated shaft joined to a head, said head being wider than said shaft;
a base member composed of a thin, flexible, skin-conformable sheet material, said base member having an upper surface and an underside, said underside being provided with an adhesive coating, said base member further comprising an opening sized larger than said stopper shaft such that said stopper shaft may pass through said opening;
a strap member having a fixed end affixed to said base member and a free end, said strap member composed of a thin, flexible, conformable sheet material, said free end of said strap member being releasably attached to said base member, said strap member having a closed status when said free end is attached to said base member and an open status when said free end of said strap member is detached from said base member, said strap member extending across said opening in said base member when in said closed status whereby said stoma stopper is retained within said opening.

7. The dressing of claim 6, wherein said strap member fixed end is affixed to said upper surface of said base member.

8. The dressing of claim 6, wherein said free end of said strap member is releasably attached to said upper surface of said base member by a hook-and-loop fastener.

9. The dressing of claim 8, wherein said hook-and-loop fastener comprises hook elements mounted on said upper surface of said base member and wherein said strap member sheet material comprises loop elements.

10. The dressing of claim 6, wherein said stoma stopper head is disk-shaped.

11. The dressing of claim 6, wherein said opening is sized smaller than said stopper head such that said stopper head may not pass through said opening.

12. The dressing of claim 6, wherein said strap member is a substantially planar member when non-tensioned.

13. A stoma stopper securement dressing comprising:
a stoma stopper comprising an elongated shaft joined to a head, said head being wider than said shaft;
a base member composed of a thin, flexible, skin-conformable sheet material, said base member having an upper surface and an underside, said underside being provided with an adhesive coating, said base member further comprising an opening sized larger than said stopper shaft such that said stopper shaft may pass through said opening;
a strap member having a fixed end affixed to said base member and a free end, said strap member composed of a thin, flexible, conformable sheet material, said free end of said strap member being releasably attached to said base member, said strap member having a closed status when said free end is attached to said base member and an open status when said free end of said strap member is detached from said base member, said strap member extending across said opening in said base member when in said closed status whereby said stoma stopper is retained within said opening.

14. The dressing of claim 13, wherein said strap member fixed end is affixed to said upper surface of said base member.

15. The dressing of claim 13, wherein said free end of said strap member is releasably attached to said upper surface of said base member by a hook-and-loop fastener.

16. The dressing of claim 15, wherein said hook-and-loop fastener comprises hook elements mounted on said upper surface of said base member and wherein said strap member sheet material comprises loop elements.

17. The dressing of claim 13, wherein said stoma stopper head is disk-shaped.

18. The dressing of claim 13, wherein said opening is sized smaller than said stopper head such that said stopper head may not pass through said opening.

19. The dressing of claim 13, wherein said strap member is a substantially planar member when non-tensioned.
